(19) United States
(12) Patent Application Publication
(10) Pub. No.: US 2021/0202109 A1
(43) Pub. Date: Jul. 1, 2021

(54) METHOD AND SYSTEM FOR REAL-TIME HEALTH DATA MANAGEMENT AND PREDICTION OF HEALTH TRENDS FOR PATIENTS

(71) Applicant: Wipro Limited, Bangalore (IN)

(72) Inventors: Venkata Subramanian JAYARAMAN, Chennai (IN); Sumithra SUNDARESAN, Chennai (IN)

(21) Appl. No.: 16/796,020

(22) Filed: Feb. 20, 2020

(30) Foreign Application Priority Data
Dec. 31, 2019 (IN) 201941054842

(51) Int. Cl. G16H 50/50; G16H 10/60; G16H 50/70; G16H 70/20; G16H 70/40; G16H 40/00; G06F 16/27; G06F 21/62; G06N 5/04; G06N 20/00

(52) U.S. Cl. CPC G16H 50/50; G16H 10/60; G16H 50/70; G16H 70/20; G06N 20/00; G16H 40/00; G06F 16/27; G06F 21/6245; G06N 5/04; G16H 70/40

(57) ABSTRACT

Disclosed herein is a method and system for real-time health data management and prediction of health trends for patients. The system receives patient data from patients, doctors treating patients and one or more external sources. Upon receiving the patient data, system generates three tables for storing, general information, health information and preference information of patients. Thereafter, the system generates a distributed ledger in real-time for each patient based on details of each patient from three tables. The distributed ledger comprises consolidated health data from all the three tables. The system provides a selectively authorized access to the distributed ledger based on biometric information of the patient. Further, based on the consolidated health data, the system manages health data of each of the one or more patients and predicts health trends of each of the one or more patients using Artificial Intelligence (AI) Machine Learning (ML) techniques.

### TECHNICAL FIELD

The present subject matter is generally related to health data processing and more particularly, but not exclusively, to a method and system for real-time health data management and prediction of health trends for patients.

### BACKGROUND

At times, patient may not recollect past information which has to be indicated to a pharmacist or a prescriber for suggesting right medication. The past information such as medications, history of diseases, allergies and the like are all important factors for a prescriber or a pharmacist to provide right medications for the patients. Due to this, the pharmacist or a prescriber may prescribe wrong medications. Further, in order to prescribe right medication, it is essential to have an appropriate mechanism for maintaining a central repository of patient data and utilizing that information for monitoring health of the patient.

At present, there is a lack of a central repository of patient history, also there is no information on patient drug consumptions and treatments from various doctors. Therefore, it is difficult to retrieve information on patient allergy, historic notes and the like. Due to lack of such information, patients may be prescribed with wrong medications which may have an adverse effect and risks life of patients.

The information disclosed in this background of the disclosure section is only for enhancement of understanding of the general background of the invention and should not be taken as an acknowledgement or any form of suggestion that this information forms the prior art already known to a person skilled in the art.

### SUMMARY

Disclosed herein is a system for real-time health data management and prediction of health trends of patients. The system comprises a processor and a memory communicatively coupled to the processor. The memory stores the processor-executable instructions, which, on execution, causes the processor to receive patient data from one or more patients, one or more doctors treating the patient and one or more external sources associated with the health data management system. Thereafter, the processor generates a first table comprising general information of each of the one or more patients based on the patient data, a second table comprising health information of each of the one or more patients based on the patient data and a third table comprising preference information of each of the one or more patients based on the patient data. Thereafter, the processor generates a distributed ledger in real-time comprising a consolidated health data for each patient based on details of each patient from the first table, the second table and the third table. Once the distributed leger is generated, the processor provides a selectively authorized access to the distributed ledger based on biometric information of the patient. Further, based on the consolidated health data, the processor manages health data of each of the one or more patients and predicting health trends of each of the one or more patients using Artificial Intelligence (AI) Machine Learning (ML) technique.

Disclosed herein is a method for real-time health data management and prediction of health trends for patients. The method comprises receiving, by a health data management system, patient data from one or more patients, one or more doctors treating the patient and one or more external sources associated with the health data management system. The method comprises generating a first table comprising general information of each of the one or more patients based on the patient data. The method also comprises generating a second table comprising health information of each of the one or more patients based on the patient data. The method further comprises generating a third table comprising preference information of each of the one or more patients based on the patient data. Thereafter, the method comprises generating a distributed ledger in real-time comprising a consolidated health data for each patient based on details of each patient from the first table, the second table and the third table. Once the distributed leger is generated, the method comprises providing a selectively authorized access to the distributed ledger based on biometric information of the patient. Based on the consolidated health data, the method comprises managing health data of each of the one or more patients and predicting health trends of each of the one or more patients using Artificial Intelligence (AI) Machine Learning (ML) technique.

Furthermore, the present disclosure comprises a non-transitory computer readable medium including instructions stored thereon that when processed by at least one processor causes the processor to receive patient data from one or more patients, one or more doctors treating the patient and one or more external sources associated with the health data management system. Further, the instructions cause the processor to generate a first table comprising general information of each of the one or more patients based on the patient data. Likewise, the instructions cause the processor to generate a second table comprising health information of each of the one or more patients based on the patient data. Further, the instructions cause the processor to generate a third table comprising preference information of each of the one or more patients based on the patient data. Also, the instructions cause the processor to generate a distributed ledger in real-time comprising a consolidated health data for each patient based on details of each patient from the first table, the second table and the third table. Further, the instructions cause the processor to provide selectively authorized access to the distributed ledger based on biometric information of the patient. Finally, the instructions cause the processor to manage health data of each of the one or more patients and predicting health trends for each of the one or more patients based on the consolidated health data of each patient using Artificial Intelligence (A) and Machine Learning (ML) techniques.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE ACCOMPANYING DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 shows an exemplary architecture for real-time health data management and prediction of health trends in accordance with some embodiments of the present disclosure;

FIG. 2a shows a block diagram of a health data management system in accordance with some embodiments of the present disclosure;

FIGS. 2b-2d shows exemplary representation of a first table, a second table and a third table respectively in accordance with some embodiments of the present disclosure;

FIG. 2e shows exemplary representation of a distributed ledger in accordance with some embodiments of the present disclosure;

FIG. 3 shows a flowchart illustrating a method for implementing KNN method for identifying new diseases, medications and health conditions based on consolidated health data of each of the one or more patients in accordance with some embodiments of the present disclosure;

FIG. 4 shows a flowchart illustrating method for real-time health data management and prediction of health trends in accordance with some embodiments of the present disclosure; and

FIG. 5 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the scope of the disclosure.

The terms “comprises”, “comprising”, “includes”, “including” or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to a method and a system for real-time health data management and prediction of health trends for patients. The system receives patient data from one or more patients, one or more doctors treating patients and one or more external sources associated with the data management system. As an example, the one or more external sources may be any management system such as pharmacy systems, Medicare systems, medical policy system which comprises information about patients. Upon receiving the patient data, the system generates three tables, namely a first table, a second table and a third table based on the patient data. The first table comprises general information of the patients which may include, but not limited to, details of a patient, details of the one or more doctors treating the patient, details of one or more pharmacy associated with the patient and details of one or more pharmacist associated with the patient. The second table comprises health information of the patients which may include, but not limited to, information of current health condition of the patient, one or more allergies of the patient and medication history of the patient. The third table comprises preference information of patients which may include, but not limited to, information of patient transfer to one or more locations, one or more preferences of patient, prescription notes provided by one or more doctors, prescription notes provided by one or more pharmacists and personal notes provided by the patient. Once the three tables are generated, the system generates a distributed ledger in real-time for each patient based on details of each patient from the first table, second table and the third table. The distributed ledger comprises consolidated health data from all the three tables. The system provides a selectively authorized access to the distributed ledger based on biometric information of the patient. If the biometric information of a user trying to access the distributed ledger matches with biometric information of the patient, then the access is provided to the user. However, if the biometric information of the user trying to access the distributed leger does not match with the biometric information of the patient, then only access to the first table and the second table may be provided to the user.

Further, the system manages health data of each of the one or more patients and predicts health trends for each of the one or more patients based on the consolidated health data of each patient using Artificial Intelligence (AI) and Machine Learning (ML) techniques.

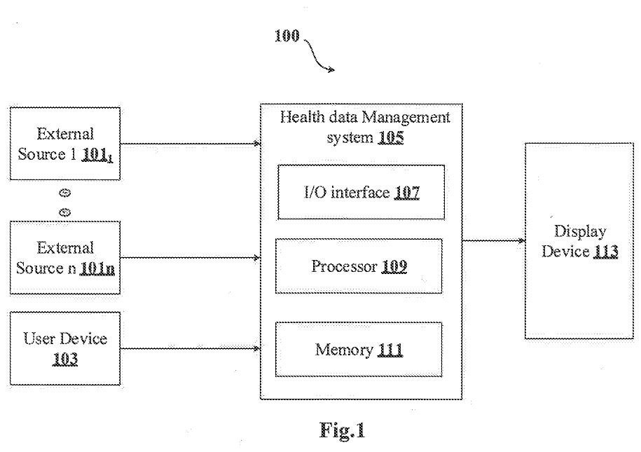

FIG. 1 shows an exemplary architecture for real-time health data management and prediction of health trends in accordance with some embodiments of the present disclosure.

The architecture 100 may include a health data management system 105 [alternatively referred as system], one or more external sources 101, a user device 103 and a display device 113. The one or more external sources 101 may be associated with the system 105. The one or more external sources 101 may include data associated with patients. As an example, the one or more external sources 101 may include, but not limited to, medical data of patients, policy data of patients and medication history. The system 105 may receive patient data from the one or more patients, one or more doctors treating the one or more patients and the one or more external sources 101. The system 105 may receive the patient data through the I/O interface 107. The received patient data may be stored in the memory 111 for further processing by the processor 109.

In an embodiment, the patient or a doctor may provide patient data using an associated user device 103. Upon receiving the patient data, the processor 109 may segregate the patient data and store in three different tables such as a first table, a second table and a third table.

In an embodiment, once the three tables are generated, the processor 109 may generate a distributed ledger. The distributed ledger may comprise storing data of the first table in a first block and storing data of the second table and the third table in a second block. The detailed explanation of storing the tables in blocks in explained in below sections. The distributed ledger may comprise a consolidated health data of each patient based on details of each patient from the first table, second table and the third table. The consolidated health data may be patient data which is stored in different systems across places where the patient is being treated. The consolidated health data may provide a consolidated perspective of patient's health in conjunction with various doctors and pharmacist accessing the data. The consolidated health data may also provide information of preferences of the patient. As an example, the preference may be preference of a doctor for treating the patient.

In an embodiment, once the distributed ledger is generated, the processor 109 may provide selective authorized access to the distributed ledger based on biometric information of the patient. If the biometric information of a user trying to access the distributed ledger matches with biometric information of the patient, then the access is provided to the user. However, if the biometric information of the user trying to access the distributed leger does not match with the biometric information of the patient, then only access to the first table and the second table may be provided to the user as the information contained in the first table and the second table may be semi-public.

Based on the consolidated health data of each patient, the processor 109 may manage health data of each patient and also predict health trends of each patient using Artificial Intelligence (AI) and Machine Learning (ML) techniques. The consolidated health data may be used by ML and AI systems to learn behavior of patients for predicting health condition of patients, medications required to cure the health conditions and predicting quantity of medicines required for treating patients. The predicted data may be displayed on the display device 113 associated with the health data management system 105.

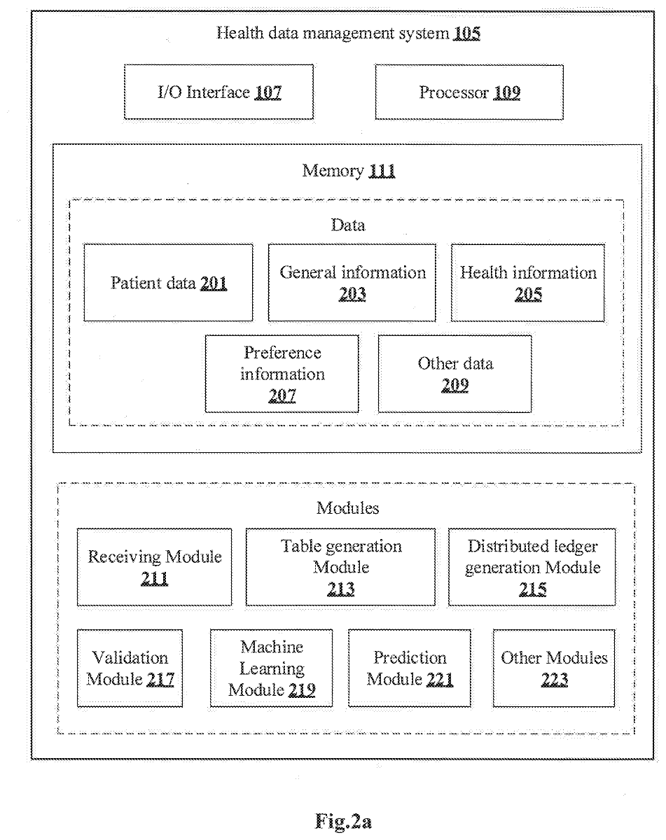

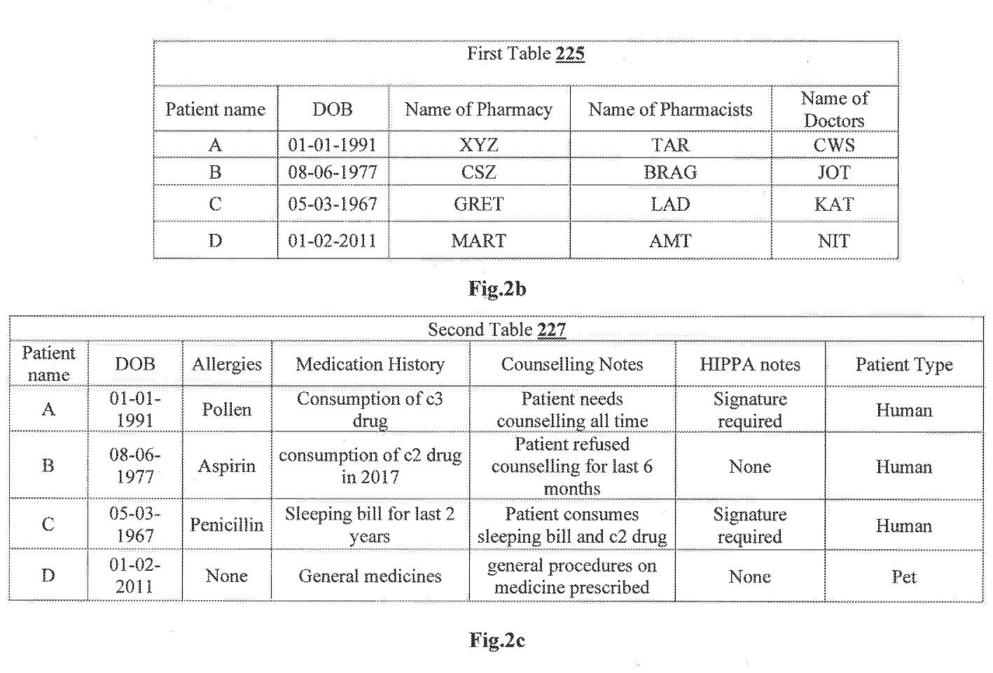

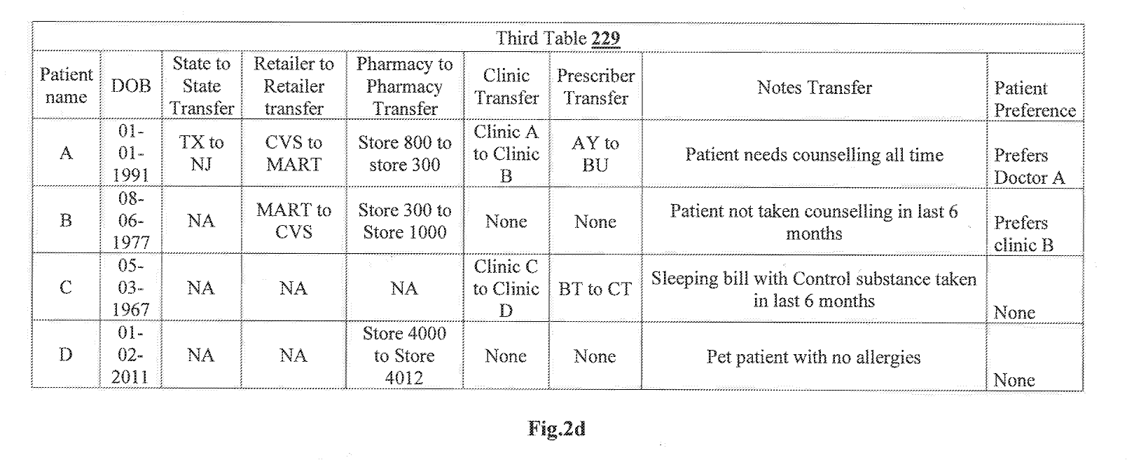

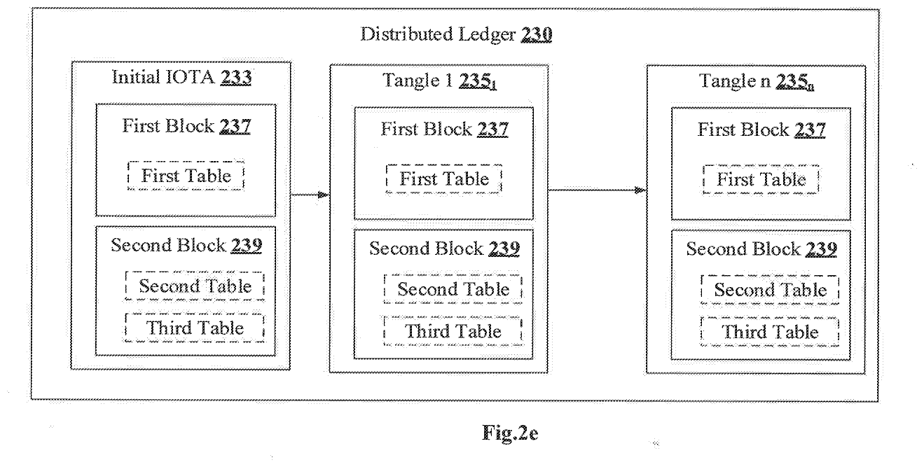

FIG. 2a shows a block diagram of a health data management system in accordance with some embodiments of the present disclosure.

In some implementations, the system 105 may include data and modules. As an example, the data is stored in a memory 111 configured in the system 105 as shown in the FIG. 2a. In one embodiment, the data may include patient data 201, general data 203, health data 205, preference data 207 and other data 209. In the illustrated FIG. 2a, modules are described herein in detail.

In some embodiments, the data may be stored in the memory in form of various data structures. Additionally, the data can be organized using data models, such as relational or hierarchical data models. The other data 209 may store data, including temporary data and temporary files, generated by the modules for performing the various functions of the system 105.

In some embodiments, the data stored in the memory 111 may be processed by the modules of the system 105. The modules may be stored within the memory 111. In an example, the modules communicatively coupled to the processor 109 configured in the system 105, may also be present outside the memory 111 as shown in FIG. 2a and implemented as hardware. As used herein, the term modules may refer to an Application Specific Integrated Circuit (ASIC), an electronic circuit, a processor 109 (shared, dedicated, or group) and memory 111 that execute one or more software or firmware programs, a combinational logic circuit, and/or other suitable components that provide the described functionality.

In some embodiments, the modules may include, for example, a receiving module 211, a table generation module 213, a distributed ledger generation module 215, a validation module 217, a machine learning module 219, a prediction module 221 and other modules 223. The other modules 223 may be used to perform various miscellaneous functionalities of the system 105. It will be appreciated that such aforementioned modules may be represented as a single module or a combination of different modules.

In an embodiment, the other modules 223 may be used to perform various miscellaneous functionalities of the system 105. It will be appreciated that such modules may be represented as a single module or a combination of different modules. Furthermore, a person of ordinary skill in the art will appreciate that in an implementation, the one or more modules may be stored in the memory 111, without limiting the scope of the disclosure. The said modules when configured with the functionality defined in the present disclosure will result in a novel hardware.

In an embodiment, the receiving module 211 may be configured to receive patient data 201 from one or more patients, one or more doctors treating the patients and from one or more external sources 101. The patients and one or more doctors may provide the patient data 201 using the associated user device 103.

In an embodiment, the table generation module 213 may be configured to generate three tables namely the first table 225, the second table 227 and the third table 229. Once the patient data 201 is received, the patient data 201 is segregated into the three tables. The first table 225 may comprise general health profile of each of the one or more patients as shown in FIG. 2b. The general health profile may be stored as general information 203. The general health profile may include, but not limited to, details of a patient such as patient name, birth date, details of the one or more doctors treating the patient, details of one or more pharmacy associated with the patient and details of one or more pharmacists associated with the patient. The second table 227 may comprise health information 205 of each of the one or more patients as shown in FIG. 2c. The health information 205 may include, but not limited to, information of current health condition of the patient, one or more allergies of the patient and medication history of the patient. The health information 205 may also include counselling notes information, Health Insurance Portability and Accountability (HIPPA) information and patient type information. The third table 229 may comprise preference information 207 of each of the one or more patients as shown in FIG. 2d. The preference information 207 may include, but not limited to, information of patient transfer to one or more locations, one or more preferences of patient, prescription notes provided by one or more doctors, prescription notes provided by one or more pharmacists and personal notes provided by the patient. The preference information 207 may also include information of pharmacy transfer, prescriber transfer, clinic transfer, retailer transfer and the like.

In an embodiment, the distributed ledger generation module 215 may be configured to generate a distributed ledger 230 in real-time which may comprise a consolidated health data of each of the one or more patients based on details of each patient from the first table 225, the second table 227 and the third table 229. In one embodiment the distributed ledger may be an open source ledger like IOTA. The distributed ledger 230 may be generated by storing the first table 225 in a first block 237 and storing the second table 227 and the third table 229 in a second block 239. This may be represented as generation of an initial IOTA 233. Further, the distributed ledger 230 may implement tangles to store the tables across the locations as shown in FIG. 2e. As shown in FIG. 2e, the distributed ledger 230 may comprise one or more tangles, tangle 1 235₁ to tangle n 235n to store the tables along with the initial IOTA 233. Each tangle may store all the three tables, the first table 225, the second table 227 and the third table 229. In an embodiment, if a modification is made to any table of any tangle, the modification may be updated in other tangles as well based on validation of biometric information provided by a user making the modifications. The validation may be performed by the validation module 217. The user may intend to modify medication of a patient. The medication information may be stored in the second table 227. The doctor may intend to modify the second table 227 in tangle 1 235₁. In order to make the modification, the user has to be authenticated. Therefore, the validation module 217 may request biometric information of the user. The biometric information of the user may be compared with pre-stored biometric information of the patient. The biometric information of the user may not match with pre-stored biometric information and hence the request for modification may be rejected. However, if a patient intends to make any modification, the biometric information of the patient may be compared with the pre-stored biometric information of the patient. Based on the match, the patient may be allowed to make any modifications. Similarly, based on biometric validation, the user may be provided access to health information 205 in the distributed ledger 230. As an example, if the user trying to access the consolidated health data from the distributed ledger 230, the biometric information of the user may be compared with biometric information of the patient by the validation module 217. If the biometric information does not match, then only access to the first table 225 and the second table 227 is provided to the user as the data in the first table 225 and the second table 227 is semi-public. However, access to the third table 229 may not be provided as preference information 207 in the third table 229 may be private only to the patient. However, if the biometric information match with the biometric information of the patient, the access to all the three tables from the distributed ledger 230 may be provided to the user. In an embodiment, the validation module 217 may provide access to the consolidated health data based on HASH data or a reference data in the distributed ledger. Based on the HASH data in the distributed ledger, the corresponding IOTA of a patient may be retrieved. Further, when a new IOTA is created for the patient, the validation module 217 secures the access to the consolidated data in the new IOTA based on biometric information and also maintains table reference in the distributed ledger.

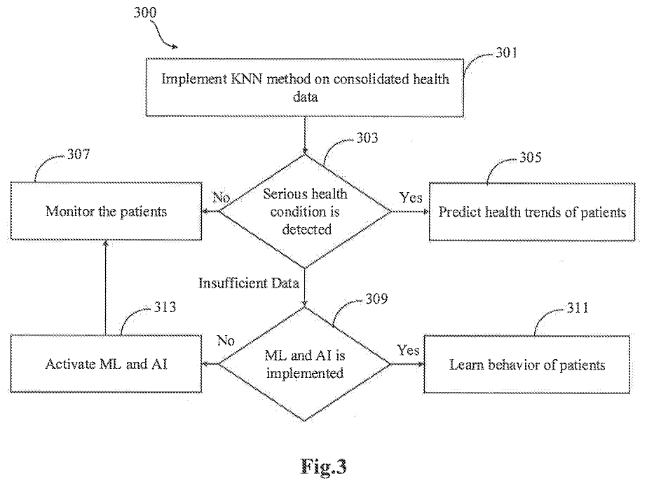

In an embodiment, the machine learning module 219 may be configured to implement K Nearest Neighbour (KNN) method to understand and identify new diseases, medications and health conditions based on consolidated health data of each of the one or more patients. FIG. 3 shows a flowchart illustrating a method of implementing KNN method for identifying new diseases, medications and health conditions based on consolidated health data of each of the one or more patients. At block 301, the KNN method may be implemented on the consolidated health data. At block 303, the method may determine if there are any serious health conditions of the patients based on the consolidated health data. If there are any serious health conditions, then the method may proceed to block 305. If there are no serious health condition, the method may proceed to block 307. At block 305, the method may predict health trends of the patients and indicate the cause for the health condition. At block 307, the method may proceed to monitor the patients. Further, if there is insufficiency of the consolidated health data for implementing KNN method, the method may proceed to block 309. At block 309, the method may determine whether the ML and A technique is active. If the ML and AI technique is active, then the method may proceed to block 311. If the ML and AI technique is not active, then the method may proceed to block 313. At block 311, the ML and AI technique may be used to learn the behaviour of the patient to make prediction of health trends of patients. At block 313, the ML and AI technique may be activated to monitor the patient continuously.

In an embodiment, the analysis of the consolidated health data may be used for prediction by the prediction module 221 which includes, but not limited to, predicting health conditions of the patients, medicines required for treating the health condition and quantity of the medicines required for treating a health condition. As an example, the consolidated health data may be analysed to provide an insight on medicines which may be relevant for group of patients based on age and the treatment which are relevant for such age of patients. The analysis of the consolidated health data may also aid in preventing occurrence of a health condition. Further, based on the analysis of the consolidated health data which provides indication of the medicines being consumed for a time period and hence may aid pharmacists to track the quantity of the medicines required for treatment. In an embodiment, the prediction module 221 may provide the predicted information to doctors, patients, and pharmacists to take necessary actions.

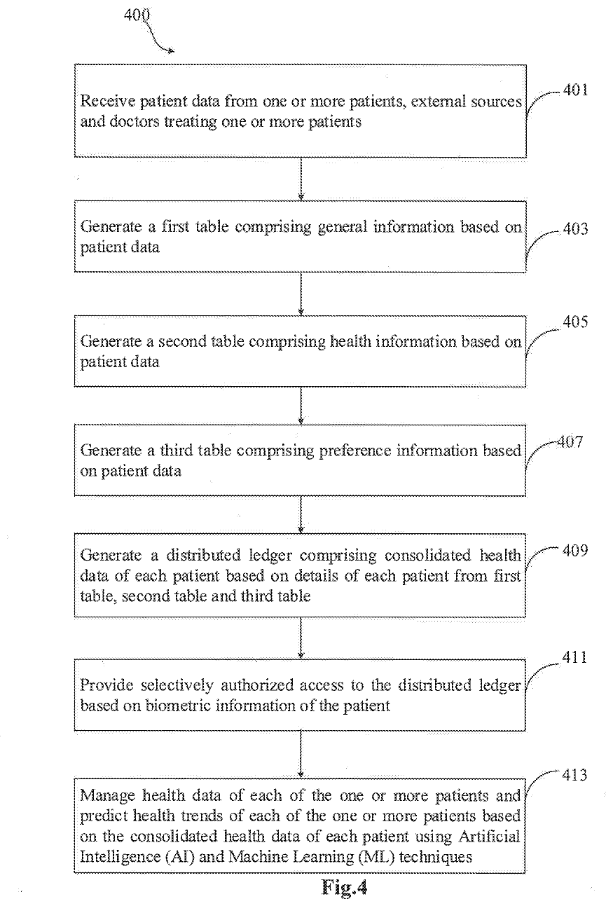

FIG. 4 shows a flowchart illustrating a method for real-time health data management and prediction of health trends in accordance with some embodiments of the present disclosure.

As illustrated in FIG. 4, the method 400 includes one or more blocks illustrating a method of. for real-time health data management and prediction of health trends. The method 400 may be described in the general context of computer executable instructions. Generally, computer executable instructions can include routines, programs, objects, components, data structures, procedures, modules, and functions, which perform specific functions or implement specific abstract data types.

The order in which the method 400 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block 401, the method may include receiving, by a health data management system 105, patient data 201 from one or more patients, one or more doctors treating the patient and one or more external sources 101 associated with the health data management system 105. The one or more external sources 101 may include data associated with patients. As an example, the one or more external sources 101 may include, but not limited to, medical data of patients, policy data of patients, medication history. As an example, the patient data 201 may include, but not limited to, name of each patient, age of each patient, contact details of each patient, patient type, names of doctors treating each patient, health condition of each patient, medication history of each patient, one or more allergies associated with each patient, biometric information of each patient, names of pharmacy associated with each patient, one or more preferences of each patient and names of pharmacist associated with each patient.

At block 403, the method may include generating, by the health data management system 105, a first table 225 comprising general information 203 of each of the one or more patients. As an example, the general information 203 may include, but not limited to, details of a patient, details of the one or more doctors treating the patient, details of one or more pharmacy associated with the patient and details of one or more pharmacist associated with the patient.

At block 405, the method may include generating, by the health data management system 105, a second table 227 comprising health information 205 of each of the one or more patients. As an example, the health information 205 may include, but not limited to, information of current health condition of the patient, one or more allergies of the patient and medication history of the patient.

At block 407, the method may include generating, by the health data management system 105, a third table 229 comprising preference information 207 of each of the one or more patients. As an example, the preference information 207 may include, but not limited to, information of patient transfer to one or more locations, one or more preferences of patient, prescription notes provided by one or more doctors, prescription notes provided by one or more pharmacists and personal notes provided by the patient.

At block 409, the method may include generating, by the health data management system 105, a distributed ledger 230. The distributed ledger 230 may comprise storing data of the first table 225 in a first block 237 and storing data of the second table 227 and the third table 229 in a second block 239. The distributed ledger 230 may comprise a consolidated health data of each patient based on details of each patient from the first table 225, second table 227 and the third table 229. The consolidated health data may be patient data 201 which is stored in different systems across the places where the patient is being treated.

At block 411, the method may include providing, by the health data management system 105, a selective authorized access to the distributed ledger 230 based on biometric information of the patient. If the biometric information of a user trying to access the distributed ledger 230 matches with biometric information of the patient, then the access is provided to the user. However, if the biometric information of the user trying to access the distributed leger does not match with the biometric information of the patient, then only access to the first table 225 and the second table 227 may be provided to the user as the information contained in the first table 225 and the second table 227 may be semi-public.

At block 413, the method may include managing, by the health data management system 105, health data of each of the one or more patients and predicting health trends of each of the one or more patients based on the consolidated health data of each patient using Artificial Intelligence (AI) and Machine Learning (ML) techniques. The consolidated health data may be used by ML systems to learn behavior of patients for predicting health condition of patients, medications required to cure the health conditions and predicting quantity of medicines required for treating patients.

### Computer System

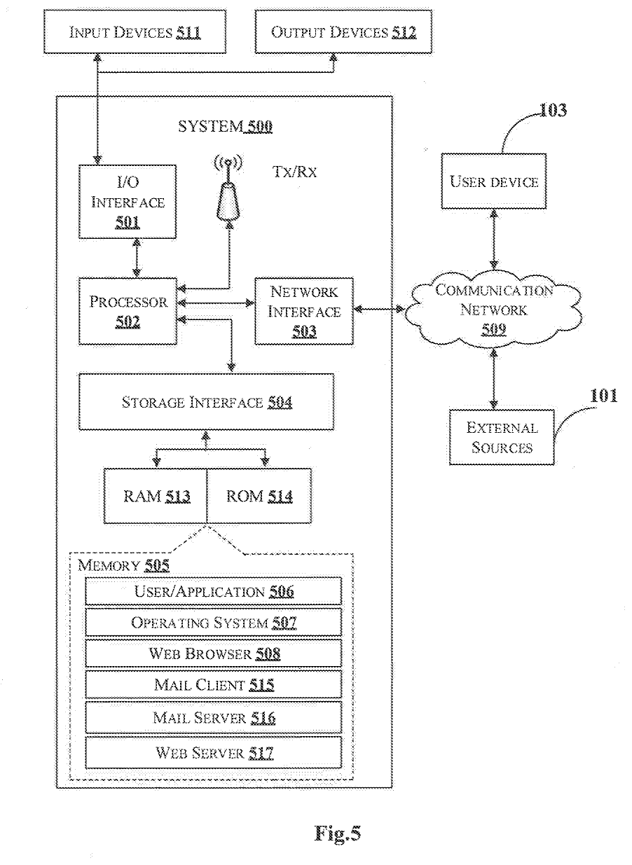

FIG. 5 illustrates a block diagram of an exemplary computer system 500 for implementing embodiments consistent with the present disclosure. In an embodiment, the computer system 500 may be a health data management system 105, which is used for real-time health data management and predicting health trends for patients. The computer system 500 may include a central processing unit (“CPU” or “processor”) 502. The processor 502 may comprise at least one data processor for executing program components for executing user or system-generated business processes. The processor 502 may include specialized processing units such as integrated system (bus) controllers, memory management control units, floating point units, graphics processing units, digital signal processing units, etc.

The processor 502 may be disposed in communication with one or more input/output (I/O) devices (511 and 512) via I/O interface 501. The/O interface 501 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc. Using the I/O interface 501, the computer system 500 may communicate with one or more I/O devices 511 and 512. The computer system 500 may communicate with the ser device 103 and the external sources 101 for receiving patient data.

In some embodiments, the processor 502 may be disposed in communication with a communication network 509 via a network interface 503. The network interface 503 may communicate with the communication network 509. The network interface 503 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communication network 509 can be implemented as one of the several types of networks, such as intranet or Local Area Network (LAN) and such within the organization. The communication network 509 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 509 may include a variety of network devices, including routers, bridges, servers, computing devices, storage devices, etc.

In some embodiments, the processor 502 may be disposed in communication with a memory 505 (e.g., RAM 513, ROM 514, etc. as shown in FIG. 5) via a storage interface 504. The storage interface 504 may connect to memory 505 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of program or database components, including, without limitation, user/application 506, an operating system 507, a web browser 508, mail client 515, mail server 516, web server 517 and the like. In some embodiments, computer system 500 may store user/application data 506, such as the data, variables, records, etc. as described in this invention. Such databases may be implemented as fault-tolerant, relational, scalable, secure databases such as Oracle® or Sybase®.

The operating system 507 may facilitate resource management and operation of the computer system 500. Examples of operating systems include, without limitation, APPLE MACINTOSH® OS X, UNIX®, UNIX-like system distributions (E.G., BERKELEY SOFTWARE DISTRIBUTION™ (BSD), FREEBSD™, NETBSD™, OPENBSD™, etc.), LINUX DISTRIBUTIONS™ (E.G., RED HAT™, UBUNTU™, KUBUNTU™, etc.), IBM™ OS/2, MICROSOFT™ WINDOWS™ (XP™, VISTA™/7/8, 10 etc.), APPLE® IOS™, GOOGLER ANDROID™, BLACKBERRY® OS, or the like. A user interface may facilitate display, execution, interaction, manipulation, or operation of program components through textual or graphical facilities. For example, user interfaces may provide computer interaction interface elements on a display system operatively connected to the computer system 500, such as cursors, icons, check boxes, menus, windows, widgets, etc. Graphical User Interfaces (GUIs) may be employed, including, without limitation, APPLE MACINTOSH® operating systems, IBM™ OS/2, MICROSOFT™ WINDOWS™ (XP™, VISTA™/7/8, 10 etc.), Unix® X-Windows, web interface libraries (e.g., AJAX™, DHTML™, ADOBE® FLASH™, JAVASCRIPT™, JAVA™, etc.), or the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### Advantages of the Embodiment of the Present Disclosure are Illustrated Herein.

In an embodiment, the present disclosure provides a method and system for real-time health data management and predicting health trends for patients.

In an embodiment, the present disclosure uses a machine learning and artificial intelligence techniques for continuous monitoring of patient data to identify new diseases, medications, and health conditions.

In an embodiment, the present disclosure provides a distributed ledger which comprises consolidated health data and the user may access the consolidated health data from across locations.

In an embodiment, the present disclosure validates access to the distributed ledger based on biometric information and hence secures access to the distributed ledger.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise. The enumerated listing of items does not imply that any or all the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference | |
|---|---|
| Number | Description |
| 100 | Architecture |
| 101 | External sources |
| 103 | User device |
| 105 | Health data management system |
| 107 | I/O interface |
| 109 | Processor |
| 111 | Memory |
| 113 | Display device |
| 201 | Patient data |
| 203 | General information |
| 205 | Health information |
| 207 | Preference information |
| 209 | Other data |
| 211 | Receiving module |
| 213 | Table generation module |
| 215 | Distributed ledger generation module |
| 217 | Validation module |
| 219 | Machine learning module |
| 221 | Prediction module |
| 223 | Other modules |
| 225 | First table |
| 227 | Second table |
| 229 | Third table |
| 230 | Distributed ledger |
| 233 | Initial IOTA |
| 235 | Tangle |
| 237 | First block |
| 239 | Second block |
| 500 | Exemplary computer system |
| 501 | I/O Interface of the exemplary computer system |
| 502 | Processor of the exemplary computer system |
| 503 | Network interface |
| 504 | Storage interface |
| 505 | Memory of the exemplary computer system |
| 506 | User/Application |
| 507 | Operating system |
| 508 | Web browser |
| 509 | Communication network |
| 511 | Input devices |
| 512 | Output devices |
| 513 | RAM |
| 514 | ROM |
| 515 | Mail Client |
| 516 | Mail Server |
| 517 | Web Server |

What is claimed is:

1. A health data management system for managing health data and predicting health trends for patients, the health data management system comprises:
a processor; and
a memory communicatively coupled to the processor, wherein the memory stores the processor-executable instructions, which, on execution, causes the processor to:
receive patient data from one or more patients, one or more doctors treating the patient and one or more external sources associated with the health data management system;
generate a first table comprising general information of each of the one or more patients based on the patient data;
generate a second table comprising health information of each of the one or more patients based on the patient data;
generate a third table comprising preference information of each of the one or more patients based on the patient data;
generate a distributed ledger in real-time comprising a consolidated health data for each patient based on details of each patient from the first table, the second table and the third table;
providing selectively authorized access to the distributed ledger based on biometric information of the patient; and
managing health data of each of the one or more patients and predicting health trends for each of the one or more patients based on the consolidated health data of each patient using an Artificial Intelligence (AI) and Machine Learning techniques.

2. The health data management system as claimed in claim 1, wherein the processor generates the distributed ledger by storing data of the first table in a first block and storing data of the second table and the third table in a second block and updates the distributed ledger when the at least one of the first table, the second table and the third table is updated.

3. The health data management system as claimed in claim 2, wherein the processor provides access to the distributed ledger when there is a match between biometric information provided by a user and the biometric information of the patient and provides access to only the first table and the second table to a user when there is a mismatch between biometric information provided by the user and the biometric information of the patient.

4. The health data management system as claimed in claim 1, wherein the patient data comprises name of each patient, age of each patient, contact details of each patient, patient type, names of doctors treating each patient, health condition of each patient, medication history of each patient, one or more allergies associated with each patient, biometric information of each patient, names of pharmacy associated with each patient, one or more preferences of each patient and names of pharmacist associated with each patient.

5. The health data management system as claimed in claim 1, wherein the general information 203 comprises details of a patient, details of the one or more doctors treating the patient, details of one or more pharmacy associated with the patient and details of one or more pharmacist associated with the patient.

6. The health data management system as claimed in claim 1, wherein the health information comprises information of current health condition of the patient, one or more allergies of the patient and medication history of the patient.

7. The health data management system as claimed in claim 1, wherein the preference information comprises information of patient transfer to one or more locations, one or more preferences of patient, prescription notes provided by one or more doctors, prescription notes provided by one or more pharmacists and personal notes provided by the patient.

8. The health data management system as claimed in claim 1, wherein the processor implements machine learning technique to learn behavior of patients using the consolidated health data for each patient in the distributed ledger for predicting health condition of the patients, medications required to cure the health conditions and predicting quantity of medicines required for treating patients.

9. A method for real-time health data management and predicting health trends for patients, the method comprising:
receiving, by a health data management system, patient data from one or more patients, one or more doctors treating the patient and one or more external sources associated with the health data management system;
generating, by the health data management system, a first table comprising general information of each of the one or more patients based on the patient data;
generating, by the health data management system, a second table comprising health information of each of the one or more patients based on the patient data;
generating, by the health data management system, a third table comprising preference information of each of the one or more patients based on the patient data;
generating, by the health data management system, a distributed ledger in real-time comprising a consolidated health data for each patient based on details of each patient from the first table, the second table and the third table;
providing, by the health data management system, selectively authorized access to the distributed ledger based on biometric information of the patient; and
managing, by the health data management system, health data of each of the one or more patients and predicting health trends for each of the one or more patients based on the consolidated health data of each patient using Artificial Intelligence (AI) and Machine Learning (ML) techniques.

10. The method as claimed in claim 9, wherein generating the distributed ledger comprises storing data of the first table in a first block and storing data of the second table and the third table in a second block, wherein the distributed ledger is updated in real-time when the at least one of the first table, the second table and the third table is updated.

11. The method as claimed in claim 9, wherein access to the distributed ledger is provided when there is a match between biometric information provided by a user and the biometric information of the patient and access to only the first table and the second table is provided to a user when there is a mismatch between biometric information provided by the user and the biometric information of the patient.

12. A non-transitory computer readable medium including instructions stored thereon that when processed by at least one processor causes the processor to:
receive patient data from one or more patients, one or more doctors treating the patient and one or more external sources associated with the health data management system;
generate a first table comprising general information of each of the one or more patients based on the patient data;
generate a second table comprising health information of each of the one or more patients based on the patient data;
generate a third table comprising preference information of each of the one or more patients based on the patient data;
generate a distributed ledger in real-time comprising a consolidated health data for each patient based on details of each patient from the first table, the second table and the third table;
provide selectively authorized access to the distributed ledger based on biometric information of the patient; and
manage health data of each of the one or more patients and predicting health trends for each of the one or more patients based on the consolidated health data of each patient using Artificial Intelligence (AI) and Machine Learning (ML) techniques.
